(19) United States
(12) Patent Application Publication
(10) Pub. No.: US 2005/0233310 A1
(43) Pub. Date: Oct. 20, 2005

(54) Screening method

(76) Inventors: Ian Hampson, Manchester (GB); Lynne Hampson, Manchester (GB)

Correspondence Address: PILLSBURY WINTHROP SHAW PITTMAN LLP, 725 S. FIGUEROA STREET, SUITE 2800, LOS ANGELES, CA, 90017, US

(21) Appl. No.: 10/506,643

(22) PCT Filed: Mar. 11, 2003
(86) PCT No.: PCT/GB03/00990

§ 371 Date: Jun. 6, 2005

(30) Foreign Application Priority Data
Mar. 12, 2002 (GB) 0205755.2

(51) Int. Cl. C12Q 1/70; G01N 33/574

(52) U.S. Cl. 435/5; 435/7.23

(57) ABSTRACT

The present invention relates to a method of screening a protein for involvement in cancer. The method involves the steps of: exposing the protein to a first viral oncoprotein; assaying for interaction of the protein and the first viral oncoprotein; exposing the protein to a second viral oncoprotein; and assaying for interaction of the protein and the second viral oncoprotein. Interaction of the protein with the viral oncoproteins indicates that the protein is involved in cancer.

The present invention relates to a method of screening a protein for involvement in cancer.

Cancer represents the second highest cause of mortality, after heart disease, in most developed countries. Current estimates suggest that one in three Americans alive at present will suffer from some form of cancer.

Many different forms of cancer exist, and it is believed that there are many different causes of the disease. Amongst the known causes of cancer are DNA damage, for example as a result of exposure to carcinogenic chemicals or radiation, and the actions of transforming viruses. It is further recognised that many cancers result from aberrant gene expression, for example as a result of abnormal levels of gene expression or of expression of mutated or otherwise altered gene products.

Although many methods for treating cancer exist, there is a well recognised need to develop new and improved techniques. Selection of a suitable treatment for cancer may be predicated on correct identification of the aetiology of the disease. For this reason it is important to identify the cause of a given patient's cancer. In addition to new treatments there is a requirement for new diagnostic tools able to detect cancers.

It is believed that there are likely to be hundreds of genes the aberrant expression of which is associated with cancer formation. Since the analysis and modulation of such genes has the potential to form the basis of both methods of diagnosis and treatment of cancers it is a recognised goal of biomedical research to identify genes, and their products, involved in oncogenesis.

Current strategies for identifying such genes and proteins are primarily based upon two strategies. The first is genomic sequencing, in which the entire genomes of the most common cancers (such as breast cancer, colon cancer, lung cancer and prostate cancer) are to be sequenced and compared with the normal human genome in order to identify mutant genes which may play a role in the development of cancer.

The second approach is to identify those genes the expression of which is altered in any particular cancer. This may be assessed by the analysis of expression levels in samples of cancerous tissue from the individual compared to expression levels in either normal tissue from the same individual or control samples taken from individuals without cancer.

Both these strategies suffer from significant drawbacks. Analysis of expression levels does not provide information regarding the presence or absence of mutations within the genes being expressed. Strategies for comparative transcript expression analysis, such as micro-array profiling, do not provide any information on cancer associated point mutations and can prove problematic when dealing with gene family members, which display areas of significant homology. The Human Cancer Genome Project will clearly identify cancer-associated point mutations but neither of the methodologies discussed will provide any direct functional annotation. Thus both approaches will identify many changes that are not causal and are merely associated with malignant disease.

Some companies currently use protein/protein interaction mapping as a general approach or alternatively retroviral expression of random peptides as a platform technology, however this technique still fails to take the functional perspective of the proteins investigated into consideration.

According to a first aspect of the present invention there is provided a method of screening a protein for involvement in cancer comprising:
i) exposing the protein to a first viral oncoprotein;
ii) assaying for interaction of the protein and the first viral oncoprotein;
iii) exposing the protein to a second viral oncoprotein; and
iv) assaying for interaction of the protein and the second viral oncoprotein
   wherein interaction of the protein with the viral oncoproteins indicates that the protein is involved in cancer.

In step i) of the invention the first viral oncoprotein is used as a “bait” to identify proteins in a library to which it is capable of binding (referred to as “prey”). A protein present in a library to be screened is thus exposed to the first viral oncoprotein under conditions in which, should the protein represent a target for the viral oncoprotein, the protein and viral oncoprotein will be able to bind to one another. Such conditions may, for example, be produced in a cell in which an interaction trap may be carried out.

Step ii) of the method allows the selection of those proteins screened that have bound to the first viral oncoprotein, the targets of the oncoprotein, based upon the interaction of the screened protein and the oncoprotein. This step may also be carried out in a suitable interaction trap.

Steps iii) and iv) of the method represent repetitions of steps i) and ii) respectively, save that in steps iii) and iv) the protein to be screened is exposed to the second oncoprotein and the relative binding of the protein and second oncoprotein assessed.

That a screened protein represents a binding partner for both the first and second oncoproteins is taken to indicate that the protein in question is involved in cancer.

The screened protein may be contained within a mixture of proteins that may or may not be involved in the aetiology of cancer.

It is preferred that those proteins that exhibit interactions with the first viral oncoprotein are identified, for instance by sequencing, and their identities noted. The same library of proteins may then be exposed to the second viral oncoprotein and the identities of those proteins interacting with the second oncogene established. Comparison of the proteins interacting with the first and second oncoproteins will allow the production of “weighted” results in which those proteins interacting with the greatest number of viral oncoproteins tested represent more favoured targets for further investigation than those interacting with lesser numbers of oncoproteins.

In an alternative embodiment only those proteins that interact with the first viral oncoprotein are exposed to second viral oncoproteins. Preferably the protein is assayed for interaction with as many of the second viral oncoproteins as are available. In this case the proteins to be screened may be exposed to the viral oncoproteins sequentially. Thus in this embodiment of the invention only those proteins that interacted with the first viral oncoprotein would be exposed to the secondary viral oncoproteins. Thus after the two rounds of screening, proteins that interact with one or more of the secondary viral oncoproteins in addition to the first viral oncoprotein would identified.

Alternatively the screened proteins may be exposed to all the viral oncoproteins (first and second) in one step. This allows identification of any protein targets that interact with more than one oncoprotein from those screened.

It will be readily appreciated that the possible number of “rounds” of screening that may be performed will only be limited by the number of viral oncoproteins available to a person effecting the invention.

Proteins identified by the screen as interacting with viral oncoproteins may be investigated for other known interactions with viral oncoproteins. This may, for example, be achieved by studying interactions reported in published literature or in relevant databases.

It will be appreciated that a given protein that shows more than one oncoprotein interaction according to the method of the invention will represent a good candidate for further investigation and validation as set out below.

It will be recognised that, in the case of multiple rounds of screening with different oncoproteins, those proteins that exhibit interactions with a greater number of oncoproteins will represent better candidates for further investigation than those proteins interacting with a lesser number of oncoproteins. The number of interactions which a given protein is deemed to exhibit may take into consideration interactions reported in, for example, published scientific literature, in addition to interactions identified by means of, for example screening using interaction traps.

Preferably the cancer is a non-viral cancer.

The tissue from which the protein is derived is preferably a tissue having a high proliferative potential, but in which the level of proliferation is low. The tissue preferably has a highly complex pattern of gene expression combined with a high capacity for proliferation. An indication of such a tissue type may be that its cells possess a relatively open chromatin structure, which is itself an indication of promiscuous low level gene expression characteristic of undifferentiated stem cells. It is preferred that the tissue is selected from the group comprising placenta, cord blood CD34⁺ haemopoietic stem cells and foetal brain. Most preferably the protein is derived from placenta or cord blood CD34⁺ haemopoietic stem cells.

The protein to be screened is preferably derived from a cDNA library. Alternatively the protein may comprise a whole cell extract or selected proteins expressed by a cell type of interest.

cDNA libraries suitable for use in the invention may be derived from any mammalian tissue. It will be appreciated that the cDNA library is ideally derived from human tissue when human genes involved in cancer are being screened.

cDNA libraries for use in the invention may be produced by any suitable method known in the prior art. Examples of suitable methods that may be used for the production of cDNA libraries are well known to those skilled in the art.

The exposure of the protein to be tested to the selected viral oncoproteins, and the assessment of their interaction, is preferably performed as part of an interaction trap. Many forms of interaction trap are known in the prior art, although it is preferred to use a yeast two-hybrid interaction trap to put the invention into effect.

Yeast two-hybrid screening is a strategy for screening for proteins that interact with a particular protein. Typically a cDNA library is constructed such that candidate proteins are expressed as translational fusions with part of a reporter gene. Yeast cells are then co-transfected with a “bait” construct consisting of the cDNA of interest fused in-frame with the other part of the reporter gene. Only if both expressed proteins physically interact will the two parts of the reporter gene be sufficiently closely associated to generate a signal.

Most preferably the yeast two-hybrid interaction trap to be used may be modified as described in Gyuris, J.; Golemis, E.; Chertkov, H and Brent, R. “Cdil, a human G1 and S phase protein phosphatase that associates with Cdk2.” (Cell. 1993 Nov. 19; 75 (4): pp. 791-803). The technique described in this paper is incorporated herein by reference. It will, however, be appreciated by those skilled in the art that any other form of interaction trap may be used to put the invention into practice. Suitable examples included techniques such as mammalian two-hybrid, bacterial two-hybrid or alternatively various types of pull down assay using, for example, an immobilised hybrid of the bait protein fused to a tag protein such as glutathione transferase (GST), or any other protein suitable for use for this purpose.

In order, when using a yeast two-hybrid interaction trap, to most efficiently assay for those yeast cells in which both bait and prey proteins are present it is necessary to selectively amplify the required yeast cells. This is independent of the presence, or lack thereof, of interactions between the proteins of interest. In prior art yeast two hybrid techniques it is usual to perform this selective amplification using agar plates which incorporate a suitable selection medium. The total number of transformed yeast cells are inoculated onto the plate which will only permit the growth of those cells that contain both the cDNA derived protein and the target protein. Such cells then produce viable colonies that are subsequently transferred to other culture plates to allow assessment of interactions.

Whilst it is advantageous to incorporate such an amplification step into the yeast two hybrid screen, there are notable drawbacks in prior art techniques in that it is difficult and time consuming to transfer the amplified colonies from the agar plates on which they have been amplified to the plates on which analysis of interactions will be performed.

We have discovered that by performing the amplification step of the yeast two hybrid screen in free solution of the suitable selection medium these disadvantages may be overcome.

The amplification of the cDNA library in yeast in free solution provides significant advantages both in terms of the ease and speed with which the yeast cells that have been positively transformed can be harvested prior to plating. Furthermore there is an increase in the efficiency of recovery of the amplified yeast cells from the culture.

Previously published methods of performing yeast two hybrid interaction screening use an inoculating loop to directly replica transfer each single colony of cells from the master screen plate to each of the plates used for growth. We have found that by spotting a defined aliquot of a colony re-suspended in a suitable diluent, such as sterile distilled water, rather than simple replica plates produced by direct colony transfer from the master plate, a defined number of cells are transferred in a precise volume per spot. The consequence of this is that subsequent growth is more uniform and can be compared in a much more precise manner.

Alternatively primary positive colonies can be inoculated into an appropriate 96 well microtitre plate and growth amplified to a uniform suspension. Replica plates can then be made using a 96 well stainless steel pin replicator and the growth of replica plates compared under different reporter gene activation conditions. Again the growth is uniform since all wells are started from the same amount of inoculum.

Oncoproteins that may be used according to the present invention may be selected from the oncoproteins expressed by any transforming virus. Preferably the first and second oncoproteins, to be used according to the present invention are selected from the group comprising oncogenic human papilloma virus (HPV), such as type 6, 16 and 18, E6, E7 and E5 proteins, hepatitis B “X”, hepatitis C “Core”, SV40 large “T” and small “T”, adenovirus “E1A” and “E1B”, human T lymphotrophic virus types 1 and 2 “Tax” proteins, Epstein Barr virus “LMP1” and “EBNA3”, JC virus large “T” and small “T”. The selected oncoproteins may be used either singly or in combinations as described above.

Preferably the first viral oncoprotein comprises HPV 16 E6.

Preferably the second viral oncoprotein comprises Tax.

In a preferred modification of the invention a protein, identified by the screen as being involved in cancer, may further be exposed to another (second) protein, or proteins, and an assay conducted for interactions between the two proteins. In such a modification proteins identified by their interaction with viral oncoproteins according to steps i) to iv) of the first aspect of the invention represent “anchor protein targets” that are then used to identify “secondary protein targets”. The secondary protein targets identified in this case are those examples of a second cellular protein that interact with the primary anchor protein target. Thus dissection of the interactions of an anchor protein target involved in cancer enables identification of further secondary protein targets that may also be involved in cancer and may represent suitable targets for further investigation as set out below.

Such secondary protein targets may, for instance, represent up or downstream members of intracellular protein networks in which the anchor protein target is involved. The interaction of the viral oncoproteins with the anchor protein target identify that the oncoproteins are able to influence the activity of the network and hence, indirectly, the activity of the secondary protein target. Examples of such networks include signalling pathways and pathways influencing gene regulation. Proteins identified in this way will represent targets for further investigation as modulators or markers of cancer.

A protein identified as an anchor protein target represents a suitable target for future investigation with respect to its involvement in cancer. However the use of these anchor protein targets as a means by which further secondary protein targets involved in cancer may be identified is a great benefit provided by the invention.

So important is this benefit that according to a second aspect of the invention there is provided a method of screening a protein sample for proteins that are secondary protein targets for viral oncoproteins comprising:
(a) exposing an anchor protein target identified as a protein involved in cancer according to the first aspect of the invention to the protein sample; and
(b) assaying for interaction of proteins within the sample with the anchor protein;
   wherein proteins identified by their interaction with anchor protein targets in step (b) represent secondary protein targets involved with cancer.

Protein samples used according to the second aspect of the invention may preferably be derived from the same tissue as the protein identified as being involved in cancer. Interactions between the proteins may be tested and assessed by means of an interaction trap, preferably by means of an interaction trap as described above.

Those proteins that are selected by any aspect of the invention as potentially involved in oncogenesis may then be sequenced in order that their identity may be discovered. Plasmids encoding the protein of interest may be extracted from cells used in the interaction trap and their sequence determined by any suitable sequencing method. Suitable means by which the plasmids may be extracted and the sequence information obtained will be readily apparent to those skilled in the art.

Knowledge of the sequence of a protein of interest, or of the gene encoding the protein, will allow searches of relevant databases to be undertaken in order to establish, where possible, the identity of the protein or gene. This identity information may then be used to investigate other reported interactions of the protein in question and also establish the function of the protein. Information regarding known interactions may allow the identification of other members of functional pathways as set out above. Information about the function of the protein may be useful in identifying the possible mode of action of the protein in oncogenesis, or in suggesting suitable means by which activity of the protein may be modulated, for instance by known modulators of a class of proteins of which the protein of interest may be a member.

A preferred embodiment would be the identification of a novel interaction of any secondary protein target with any of the viral oncoproteins listed thus picking out the particular anchor/secondary protein target pathway as being the target of multiple viral insults. Ideally the new secondary protein target would also be present in the newly acquired catalogue of anchor protein targets.

Bioinformatic analysis of proteins identified according to the first or second aspects of the invention and also of pathways identified by the screen as being involved in cancer may be carried out as follows. Genes extracted from plasmids encoding anchor or secondary protein targets can be identified by the use of BLAST search of the non-redundant and “Expressed Sequence Tag” (EST) NCBI nucleotide databases. This strategy can also be used to identify other gene family members. Expression profiles can be evaluated in silico by the use of SAGE tag virtual Northern Blots. Information regarding reported functions and known interactions of targets identified by the screen as involved in cancer may be contained in scientific publications or other public domain databases. These can be accessed via Pubmed, Unigene Gene Cards as can information regarding the chromosomal location of targets. Chromosomal location can also be ascertained by use of the Human Genome Gateway BLAT search program which provides information regarding intron/exon boundaries, gene structure, identity of adjacent genes and available ESTs together with access to gene prediction programs such as ENSEMBL. Chromosomal locations can be evaluated for cancer associated amplifications/deletions etc. by the use of the Pubmed database. The Pubmed data base can also be used to identify any secondary protein targets previously shown to interact with an anchor protein target under investigation. For example searches can be performed incorporating the name of the prospective target and that of a suitable interaction trap method. Alternatively the names of additional viral oncoproteins can be incorporated as search terms along with the names of either anchor protein targets or secondary protein targets identified using specific oncoprotein baits, or the genes encoding such targets. Using PubMed all prospective “anchor” and “secondary” targets can be cross referenced with the broad functional names of categories of protein that are known to be responsive to the action of commonly available “drugs”. Examples of classes of target proteins for which a range of existing drugs are available include proteases, kinases, phosphatases, ion channels etc.

Homology comparison of two sequences can be carried out using pairwise BLAST (NCB). If the gene, and hence target protein, function is unknown this can be evaluated by using protein/protein cross species homology searches to such organisms as C. Elegans, D. Melanogaster, S. Cerevisiae, or M. Musculus. Cross species interlogs can also be used to identify additional potential interactors within common pathways.

Knowledge of the function of a protein of interest may represent another criteria by which favoured targets for further investigation or validation may be identified. For example it may be preferred to investigate those proteins of a certain class, or for which known modulators exist, as a matter of priority.

Information regarding the sequence of plasmids encoding those proteins identified by the interaction trap as potentially of interest is also useful in discounting coding sequences that may give “false positive” results. Such coding sequences may, for example, be those located in untranslated regions or other genomic contaminants.

In a preferred embodiment the method of the invention further comprises a validation step. Proteins identified by the method of the invention as involved with cancer, or the nucleic acids encoding such proteins, represent suitable “targets” for such validation.

Such a validation step may, for example, comprise analysis of expression levels of targets identified by the screen. Analysis of levels of expression of identified targets, and also of mutated forms of identified targets may preferably be conducted by means of comparing the degree of expression of gene products, and the identity of the products expressed, between samples of cancerous and non-cancerous tissues. Preferably such tissue samples will be derived from the same tissue types, and most preferably the cancerous and non-cancerous tissue samples will be derived from the same individual.

Analysis of expression of targets identified by the method of screening of the invention may also include analysis of expression of mutant forms of targets identified. By “mutant forms” we mean any proteins having at least 50% sequence homology (i.e. the sequence of the amino acids forming the protein or of the nucleic acids encoding the protein) with the targets identified by the screening method. Preferably mutant forms of the target will share at least 75% homology with the wild-type target, and most preferably mutant forms of the target share at least 90% homology with the wild-type target gene or gene product.

In an alternative validation step mutant forms of nucleic acids encoding target proteins identified by the screening method may be evaluated and analysed by any suitable technique known in the prior art. For example, such analysis may be performed by multiplex PCR Multiplex PCR may, for example, be performed on a real time multiplex PCR machine. Alternatively RNA expression and altered splice forms can also be evaluated by Northern Blots. Comparison of expression levels can be carried out using matched pair tumour/normal total cDNA array blots such as those produced by Clontech.

A suitable validation step may also comprise analysis of the presence of point mutations in the genes encoding proteins identified by the screening method. A suitable method by which such an investigation may be carried out is by assessment of denaturing HPLC (Transgenomic Wave analysis) studies comparing cDNA amplification products derived from cancerous and non-cancerous tissue samples of the type indicated above. Other sequencing methods known in the art are also suitable for use in analysis of putative mutations in genes encoding target proteins.

Mutations of genes encoding targets identified by the screening method of the invention may also be detected by analysis of the genomic DNA from which the cDNA referred to above are derived.

Another parameter that may be investigated in any validation step used may be “allelic loss” or “homozygous loss” among genes encoding target proteins identified by the screen of the invention Homozygous loss occurs when a specific gene has deletions present in both alleles. Homozygous loss can be detected by PCR of either genomic or cDNA. Other suitable techniques that may be employed are Northern or Southern blotting. Allelic loss occurs when only one allele of a gene is deleted. Allelic loss can be demonstrated by analytical PCR comparison of genomic DNA from normal and tumour tissues using an informative single nucleotide polymorphism (SNP), identified from the public database, or a designed SNP assay. Allelic loss is indicated by the presence of a restriction fragment polymorphism in one allele and not the other.

Validation of the interactions and properties of both target proteins and their coding genes may also be investigated in vitro or in vivo.

For example genes encoding targets may be cloned into vectors expressing detectable “tags” such as the pcDNAV5His vector. Expression of a gene of interest using this vector causes the protein to be produced bearing a small antigenic “tag” protein V5 that facilitates the immuno-localisation of the target protein. Thus expression of targets in conjunction with such tag proteins is particularly suited to the use of immuno-precipitation studies to investigate the interactions of the target protein and other molecules, such as viral oncoproteins or other members of putative pathways, in cultured cells.

Proteins identified according to the first or second aspects of the invention as being involved with cancer, i.e. to say anchor protein targets and secondary protein targets, represent targets for therapeutic or diagnostic intervention.

Further information as to the effect of over-expression of a protein identified according to the first or second aspects of the invention, or alternatively blockade of its expression, may be obtained by, for example gene transfer experiments or the use of anti-sense or siRNA oligonucleotides. Such experiments may preferably be undertaken in wide variety of transformed or non-transformed cell lines depending on the activity that it is sought to assess. Effects on cell growth, contact inhibition, altered ability to undergo anchorage independent growth and apoptosis may be typically measured. Suitable systems by which gene expression may be induced include the Invitrogen GeneSwitch system in which induction of gene expression may be brought about by exposure to mifepristone. In vivo analysis of the effects of expression of target proteins and their possible interactions may be effected using well known techniques such as in vivo tumour xenograft models.

A protein that is indicated, by the methods of the invention, to be involved with cancer may be selected as a target for modulation for therapy for cancer, or as a marker for the diagnosis of cancer.

Once targets have been identified by means of the screening method of the invention then compounds that may be used to provide novel cancer therapies based upon the manipulation of levels or activity of the target may be found. Such compounds may, for instance, be compounds that influence the level of transcription of the gene encoding the target, or influence the accumulation or bio-availability of the target.

The compounds may be used to treat cancer as a monotherapy (i.e. use of the compound alone) or in combination with other compounds or treatments used in cancer therapy (e.g. chemotherapeutic agents, radiotherapy).

Known procedures, such as those conventionally employed by the pharmaceutical industry (e.g. in vivo experimentation, clinical trials etc), may be used to establish specific formulations of medicaments and precise therapeutic regimes (such as daily doses of the agents and the frequency of administration) that may be used to influence the expression of identified targets and thereby provide novel therapies for cancers in which the target is implicated.

Such novel therapies may be used for the purposes of treating an existing cancer or may be used as a prophylactic treatment administered to a person believed to be at risk of developing such a cancer.

Once targets have been identified it will be appreciated that such targets may be used as the basis for methods for the diagnosis of cancer. Such methods may, for example, comprise analysing a cell sample from a patient for the presence of a mutant form of the target and/or altered expression of the wild type target.

Diagnosis may be effected on a sample from a patient believed to be suffering from cancer, or alternatively to a patient believed to be at risk of developing cancer.

Diagnosis according to the invention may be effected in order to establish whether or not a patient is suffering from cancer. As an alternative diagnosis may be effected in order to assess the suitability of a specified therapeutic regime for the treatment of a patient's disease.

The sample taken may, for instance, be a tissue biopsy, blood sample or swab.

Diagnosis of the cellular levels of either the wild-type or mutant forms of the target may be carried out by assessing the level of the product of the target within the cell, or alternatively by taking a measurement of the level of transcription of the gene encoding the target. The expression of wild-type or mutant products of the target may, for example, be assessed through the use of specific binding agents including polyclonal and monoclonal antibodies in techniques such as immuno-cytochemistry, immuno-precipitation, immuno-blotting (Western blotting) or enzyme linked immuno-sorbent assay (ELISA).

Detection of the wild type or mutant form may be directed to either detection of the protein, or detection of the genetic material encoding the wild-type or mutant protein.

A preferred experimental protocol for effecting the first aspect of the invention is as follows:

Steps i) to iv):

Proteins encoded by a suitable cDNA library, such as one derived from placenta or foetal brain, are screened by a yeast two-hybrid methodology (Gyuris et al., supra) with selected oncoproteins, such as HPV 16 E6 and “Tax”, as both 5′ and 3′ lexA fusions. A Qbot (Genetix) is used to facilitate the screening process. The results of this screen are a 96 well format panel of yeast clones, which express the putative protein prey for each viral oncoprotein. Duplicate multiwell plates are inoculated and screened by blue/white X-Gal selection for interactions between the proteins being screened and the viral oncoproteins.

Sequencing of Genes Encoding Proteins Identified by the Method of the Invention as Being Involved in Cancer:

Colonies exhibiting positive interactions between proteins being screened and the viral oncoproteins are picked into 2 ml deep well 96 well plates, expanded in culture and cDNA “prey” inserts amplified from a small aliquot of this culture by the use of vector flanking primers. All preys are preferably PCR sequenced by use of an automated DNA sequencer (eg ABI3100) and cDNA inserts identified by BLAST search Alternatively, so called “smash and grab” yeast plasmid preparations can be prepared from each 2 ml deep well yeast culture and used to transform E. Coli KC8 cells growing on M9 minimal medium plus essential amino acids, minus tryptophan. Colonies obtained after approximately two days contain the tryptophan, auxotroph library plasmid that contains the putative prey interactor. These colonies will either be used to PCR amplify the library prey insert directly or plasmid can be amplified by expansion of the colony in liquid culture and plasmid purification carried out. Both of these approaches provide prey material that can be seqeunced as previously described.

Bioinformatics Analysis of Genes Encoding Proteins Identified by the Method of the Invention as being Involved with Cancer:

This provides the identity of all candidate genes, thereby allowing identification of those potential “anchor” target preys that are common to more than one viral oncoprotein and allows grouping into categories according to gene function by the use of bio-informatics (eg NCBI data base, PubMed, Human Genome Gateway etc.) It also allows elimination of; sibling clones derived from the same parent cDNA; clones derived from non-coding sequence and out of frame sequence. Those protein preys common to more than one oncoprotein, are the first to be selected for target validation studies. Some protein preys may not directly interact with more than one oncoprotein but may represent strong candidates since there may be alternative “secondary” targets identified within a common pathway that interact with additional viral oncoproteins to produce a similar oncogenic effect. Thus it is also important to establish the possibility of multiple viral oncoprotein involvement in common cellular pathways. The result of this analysis is the identification of putative protein targets within an interaction cascade from the protein identified according to the first aspect of the invention.

A preferred protocol for effecting the second aspect of the invention is as follows:

The steps i) to iv) identified above were performed to identify anchor protein targets for use in the second aspect of the invention. The following further steps are also performed.

Second round yeast two hybrid screens may be carried out using selected anchor protein targets as bait The products of these screens represent potential new secondary protein targets, which are then compared to the newly identified catalogue of anchor protein targets of the different viral oncogenes studied. These secondary protein targets are also functionally evaluated by the use of bio-informatics for in silico interaction mapping. On the basis of these findings, targets are then prioritised for further validation studies according to “druggability” (e.g. ion channel, kinase, proteases etc.) and the number of viral oncogenes that target a particular interaction cascade. If the function of any novel target is unknown it is possible to gain clues to this by examining the database of other species such as c. elegans, S. cerevisiae, D. melanogaster etc for homologous sequences since there may be functional data on these homologues. Thus a matrix of weighted results of those cellular proteins and their pathways and the number of viral oncoproteins that interact with the pathway is produced.

Validation steps that may be used according to the first and second aspects of the invention are as follows:

Validation Phase One:

Selected “anchor” and “secondary” target genes are fed directly into a detailed analysis of transcript expression (Multiplex PCR), mutation (Denaturing HPLC, Sequencing), allelic loss etc. in a variety of human cancers and corresponding normal tissue types isolated, wherever possible, from the same patient. These data also provide a basis for distinguishing between targets that are associated with viral life cycle and those that are associated with oncogenesis. This phase of the analysis may be performed using a real time multiplex PCR machine such as the MX4000 (Stratagene).

Validation Phase Two:

Based on validation phase one findings, selected targets are entered into a program of studies using a variety of cell culture systems. Cloning into “Tag” vectors, transient transfection and immuno-precipitation are used to confirm oncogene-target interaction in mammalian cells. Gene transfer and the use of anti-sense or siRNA oligonucleotides mediated gene silencing are used to assess the effects of either over expression or blocking expression of selected targets in a variety of different transformed and non-transformed cell lines. Gene silencing experiments are particularly valuable since they identify potential gain-of-function targets that are necessary for the malignant characteristics of transformed cells. pSwitch transformed and non-transformed cell lines may be used with the Gene Switch (Invitrogen) mifepristone inducible system for evaluating the effects of stable induced expression of targets in vitro. The same system can be used for controlled gene expression with a murine in vivo tumour xenograft model.

The invention will now be described, by way of example only, with reference to the accompanying example and figure in which:

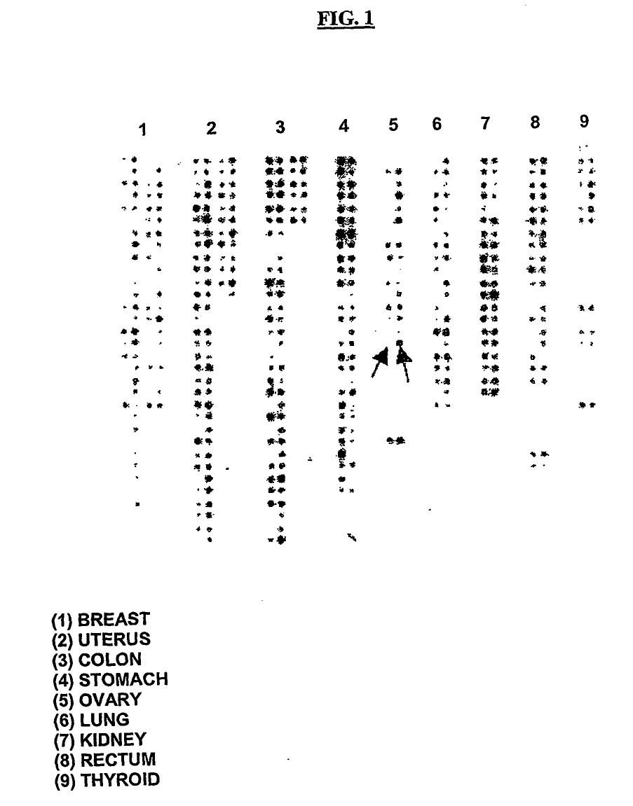

FIG. 1 represents the results of probing a panel of pair matched cDNAs from normal and tumour tissue using radioactively labelled TIP-1 coding sequence.

### EXAMPLE

Yeast two-hybrid selection, as described by Gyuris et al., supra, was used to identify proteins derived from a human placenta cDNA library that bound to the viral oncoproteins human papilloma virus type 6 E6 and human T-cell leukaemogenic virus ‘Tax’.

Yeast cells expressing proteins derived from the placenta cDNA library were first exposed to HPV 16 E6 and those clones expressing human proteins that interacted with the oncoproteins noted. The same panel of yeast cells expressing the cDNA library were then assayed for interactions with “Tax”, and those clones that interacted with the oncoprotein noted Comparison of the lists of interacting clones produced allowed those clones that encoded human proteins that reacted to both oncoproteins to be identified.

The gene encoding one of those placenta derived proteins that exhibited interactions with both viral oncoproteins was sequenced after prey plasmid extraction and found to have a sequence as follows (Sequence ID No. 1):

| | AGGGGCGCTC CGGCCAGTGA TTGGCTGGAG GTTTGTTAAC TATTCATGAG GGGGCGGGCC |
|---|---|
| 61 | GAGCGGGGCG GCCTTTGTTA AGCAGCGAGG GCGCGACCGC GGGTACTCTG CTGCCGGCTT |
| 121 | CTCGGAGCGG CGCTGGGCGA CCAGAGCAGG GTCGAGATGT CCTACATCCC GGGCCAGCCG |
| 181 | GTCACCGCCG TGGTGCAAAG AGTTGAAATT CACAAGCTGC GTCAAGGTGA GAACTTAATC |
| 241 | CTGGGTTTCA GCATTGGAGG TGGAATCGAC CAGGACCCTT CCCAGAATCC CTTCTCTGAA |
| 301 | GACAAGACGG ACAAGGGTAT TTATGTCACA CGGGTGTCTG AAGGAGGCCC TGCTGAAATC |
| 361 | GCTGGGCTGC AGATTGGAGA CAAGATCATG CAGGTGAACG GCTGGGACAT GACCATGGTC |
| 421 | ACACACGACC AGGCCCGCAA GCGGCTCACC AAGCGCTCGG AGGAGGTGGT GCGTCTGCTG |
| 481 | GTGACGCGGC AGTCGCTGCA GAAGGCCGTG CAGCAGTCCA TGCTGTCCTA GCAGCCACCA |
| 541 | CCATCTGCGA CTCCTGCCTG CCGCCTCTCT GTACAGTAAC GCCACTTCCA CACTCTGTCC |
| 601 | CCATCTGGCT TCTGCTGACC GCTGGGCCCC AGCTCAGAAG GGCTATAGCT GGTCCCAGAG |
| 661 | GCTTGGCCTG GCCTTCCTTC CCTTCTCCCA TCCCTGGCCT GGGGCCTCTG GGACCAGCTT |
| 721 | TCTCTCCTGG ACACCGAGGA TTGGAAATAA GGGCCTGGAG CTGAGTAGTA GCCAGTCTGC |
| 781 | TGTGACCACA GGCTCAGGTC CGACCCTGCT GCTTGGCCAC AGCAGTGGCT GGGCAAGTGG |
| 841 | GAACCACTAT CTCTTGGGAG CCCCCAAAAG CTGGGAAATG CTGGAGGAAC CAGGCCTTTC |
| 901 | CCGCTTTTGC CTGGCTGCAG GGTTCGGCTC CGCCCCTGCC CCCCAGCCCT CGTGTGTCCA |
| 961 | CACCGCAGTG CCTCTGCCCC TCGGGGGACT GGACACACAT CCTGCCAGAG GCGCTACGAA |
| 1021 | GCTTTGCCCA GATGAAGCCA GGTGGGCTCC GCGTTCACTC CCACTCTCCC GAGGGGTGCT |
| 1081 | GGCCTCCCCA GGGTTTGCCT TCTTACGGAT TTAGACGAGG TTCGAGGCTC ACCTATCAGG |
| 1141 | GCAGCTCTCA GGATTGTCAT TTTCCTCTTT GCCTGTGGGT TTAACTTTTG TATTTTTTTA |
| 1201 | ATCACAAGTT TGATACAAAA TGTTTTTATC GTACTCTTTG GAGATGCCCA TTCTACTTTT |
| 1261 | GAATTTAGCT TTTACTAATT CGCATCTGGA AGCTCAGCAA GTGCACAAGC CTTACTTTGG |
| 1321 | TTACCGTGGA AACCACTGCC GCCCCTCCCC GATGTGGTGC GCTCAATAAA AATGCTGGAA |
| 1381 | TTCAAAAAAA |

Comparison of sequence ID No. 1 with publicly available database information revealed that sequence ID No. 1 corresponded to the published sequence of the gene (Accession number AF028823) encoding the HTLV “Tax” interacting protein 1 (TIP-1).

In order to validate the method and identify that TIP-1 is indeed involved in cancer full length Tip-1 coding sequence was radioactively labelled by random priming with α³²P dCTP and used to screen a panel of 250 matched pairs of normalised total cDNAs from normal (Left hand column dots in FIG. 1) and tumour tissue (Right hand column dots FIG. 1) from the same individual. (Clontech). (See FIG. 1). This blot covers a wide range of human cancer types and the results indicated that the expression of TIP-1 was up-regulated approximately ten fold in; 28% of (n=14) ovarian carcinoma, 36% of (n=50) breast carcinoma, 33% of (n=21) lung carcinoma, 30% of (n=42) in uterine carcinoma and 50% of (n=6) in thyroid carcinoma However, analysis of the histology from these various carcinomas indicated that different carcinomas, such as lung, had sub-classifications within the overall category. Out of 21 lung carcinomas 5 were keratinising, of which, non showed any up-regulation of Tip-1. Out of the remaining 16 lung carcinomas, 8 (50%) showed extensive up-regulation of Tip-1 expression.

These results are consistent with TIP-1 being involved in cancer. The fact that TIP-1 was identified by following methods according to the first aspect of the invention validates the claimed method.

1. A method of screening a protein for involvement in cancer, comprising:
i) exposing the protein to a first viral oncoprotein;
ii) assaying for interaction of the protein and the first viral oncoprotein;
iii) exposing the protein to a second viral oncoprotein; and
iv) assaying for interaction of the protein and the second viral oncoprotein,
wherein interaction of the protein with the viral oncoproteins indicates that the protein is involved in cancer.

2. The method according to claim 1, wherein only those proteins that interact with the first viral oncoprotein are exposed to the second viral oncoprotein.

3. The method according to claim 1, wherein the protein is contained within a mixture of proteins which may or may not be involved in the aetiology of cancer.

4. The method according to claim 1, wherein the protein is derived from a tissue having a highly complex pattern of gene expression combined with a high capacity for proliferation.

5. The method of claim 4 wherein the tissue is selected from the group consisting of placenta, cord blood CD34⁺ haemopoietic stem cells, and foetal brain.

6. The method according to claim 1, wherein those proteins that exhibit interaction with a viral oncoprotein are correlated with interactions with other oncoproteins.

7. The method according to claim 1, wherein the first and second viral oncoproteins are independently selected from the group consisting of human papilloma virus type 6, 16 and 18, E6, E7 and E5 proteins, hepatitis B “X”, hepatitis C “Core”, SV40 large “T” and small “T”, adenovirus “E1A” and “E1B”, human T lymphotrophic virus types 1 and 2 “Tax”, Epstein Barr virus “LMP1” and “EBNA3”, and JC virus large “T” and small “T”.

8. The method according to claim 7, wherein the first oncoprotein is HPV 16 E6.

9. The method according to claim 7, wherein the second oncoprotein is “Tax”.

10. The method according to claim 1, wherein the protein is derived from a cDNA library.

11. The method according to claim 10, wherein the primary sequence of the protein can be derived by cross reference to the nucleic acid base sequence of the parent cDNA.

12. The method according to claim 1, additionally comprising a validation phase.

13. The method according to claim 12, wherein the validation phase comprises at least one of the following further steps:
a) analysing expression levels of a protein, indicated as being involved in cancer, in cancerous and non-cancerous tissue samples;
b) comparing the levels of expression of the protein in the cancerous and non-cancerous tissues;
c) analysing the effects of targeted antisense or siRNA oligonucleotide mediated gene silencing on the growth characteristics of transformed and non-transformed cell lines;
d) analysing the effects of constitutive or inducible expression of proteins in transformed and non-transformed cell lines;
e) analysing the effects of antibodies or intrabodies directed at one or more domains of the protein; and
f) comparing the primary amino acid sequence of the protein or parent nucleic acid base sequence with one or more databases of known sequences of other proteins to identify homology with functional domains and infer putative involvement in cancer.

14. The method according to claim 12, wherein the validation step comprises analysis of nucleic acids derived from a tissue sample, tumour biopsy or cell line for the presence of mutant forms of the nucleic acids encoding a protein indicated as being involved in cancer.

15. The method according to claim 12, wherein the validation phase comprises analysis of DNA derived from a tissue sample, tumour biopsy, or cell line for the presence of mutant forms of the DNA encoding a protein indicated as being involved in cancer.

16. The method according to claim 1, further comprising:
v) exposing a protein identified as a protein involved in cancer to a protein sample; and
vi) assaying for interaction of proteins within the sample with the protein identified as involved in cancer,
wherein proteins identified in step vi) are secondary protein targets involved with cancer.

17. The method according to claim 16, wherein the second protein is derived from the same tissue as the protein exposed to the viral oncoproteins.

18. The method according to claim 1, wherein the protein indicated to be involved with cancer is selected as a target for modulation for therapy for cancer.

19. The method according to claim 1, wherein the protein indicated to be involved with cancer is selected as a marker for the diagnosis of cancer.

20. A method of screening a protein sample to identify proteins that are secondary protein targets for viral oncoproteins comprising:
i) exposing protein identified as a protein involved in cancer according to claim 1 to the protein sample; and
ii) assaying for interaction of proteins within the sample with proteins identified by their interaction with viral oncoproteins according to claim 1;
wherein proteins identified by their interaction with the protein involved in cancer in step ii) represent secondary protein targets involved with cancer.

21. The method of claim 20, further comprising the step of:
iii) investigating the functional validation of the secondary targets in cancer by at least one of the following further steps:
a) analysing expression levels of a protein, indicated as being involved in cancer, in cancerous and non-cancerous tissue samples;
b) comparing the levels of expression of the protein in the cancerous and non-cancerous tissues;
c) analysing the effects of targeted antisense or siRNA oligonucleotide mediated gene silencing on the growth characteristics of transformed and non-transformed cell lines;
d) analysing the effects of constitutive or inducible expression of proteins in transformed and non-transformed cell lines;
e) analysing the effects of antibodies or intrabodies directed at one or more domains of the protein; and
f) comparing the primary amino acid sequence of the protein or parent nucleic acid base sequence with one or more databases of known sequences of other proteins to identify homology with functional domains and infer putative involvement in cancer.


SEQUENCE LISTING (1 sequence)

SEQ ID NO: 1 - DNA, 1390 bp, Homo sapiens
aggggcgctccggccagtgattggctggaggtttgttaactattcatgagggggcgggccgagcggggcggcctttgttaagcagcgagggcgcgaccgcgggtactctgctgccggcttctcggagcggcgctgggcgaccagagcagggtcgagatgtcctacatcccgggccagccggtcaccgccgtggtgcaaagagttgaaattcacaagctgcgtcaaggtgagaacttaatcctgggtttcagcattggaggtggaatcgaccaggacccttcccagaatcccttctctgaagacaagacggacaagggtatttatgtcacacgggtgtctgaaggaggccctgctgaaatcgctgggctgcagattggagacaagatcatgcaggtgaacggctgggacatgaccatggtcacacacgaccaggcccgcaagcggctcaccaagcgctcggaggaggtggtgcgtctgctggtgacgcggcagtcgctgcagaaggccgtgcagcagtccatgctgtcctagcagccaccaccatctgcgactcctgcctgccgcctctctgtacagtaacgccacttccacactctgtccccatctggcttctgctgaccgctgggccccagctcagaagggctatagctggtcccagaggcctggcctggccttccttcccttctcccatccctggcctggggcctctgggaccagctttctctcctggacaccgaggattggaaataagggcctggagctgagtagtagccagtctgctgtgaccacaggctcaggtccgaccctgctgcttggccacagcagtggctgggcaagtgggaaccactatctcttgggagcccccaaaagctgggaaatgctggaggaaccaggcctttcccgcttttgcctggctgcagggttcggctccgcccctgccccccagccctcgtgtgtccacaccgcagtgcctctgcccctcgggggactggacacacatcctgccagaggcgctacgaagctttgcccagatgaagccaggtgggctccgcgttcactcccactctcccgaggggtgctggcctccccagggtttgccttcttacggatttagacgaggttcgaggctcacctatcagggcagctctcaggattgtcattttcctctttgcctgtgggtttaacttttgtatttttttaatcacaagtttgatacaaaatgtttttatcgtactctttggagatgcccattctacttttgaatttagcttttactaattcgcatctggaagctcagcaagtgcacaagccttactttggttaccgtggaaaccactgccgcccctccccgatgtggtgcgctcaataaaaatgctggaattcaaaaaaa